(19) United States
(12) Patent Application Publication
(10) Pub. No.: US 2023/0196057 A1
(43) Pub. Date: Jun. 22, 2023

(54) METHOD AND SYSTEM FOR REMEMBERING ACTIVITIES OF PATIENTS WITH PHYSICAL DIFFICULTIES AND MEMORIES OF THE DECEASED ON METAVERSE PLATFORM

(71) Applicant: Korea Electronics Technology Institute, Seongnam-si (KR)

(72) Inventors: Jin Woong CHO, Hanam-si (KR); Myoung Ko, Hanam-si (KR)

(73) Assignee: Korea Electronics Technology Institute, Seongnam-si (KR)

(21) Appl. No.: 17/986,135

(22) Filed: Nov. 14, 2022

(30) Foreign Application Priority Data
Dec. 17, 2021 (KR) 10-2021-0181562

(51) Int. Cl. G06N 3/00; G06T 13/40

(52) U.S. Cl. CPC G06N 3/006; G06T 13/40

(57) ABSTRACT

There are provided a method and a system for operating a metaverse platform, which operate to remember activities of patients with physical difficulties and memories of the deceased. According to an embodiment of the disclosure, a metaverse platforming operating system includes: a data collection unit configured to collect image and voice data of a user; a voice and image correction module configured to correct an image and a voice of the user by inputting the collected image and voice data to a trained AI model; an avatar generation module configured to generate a virtual avatar reflecting a facial image of the user in a metaverse, based on the corrected image; and an avatar action control module configured to control an action of the generated avatar according to an inputted input signal. Accordingly, patients, family, and acquaintances may smoothly communicate with patients with physical difficulties in a metaverse virtual world, and may remember memories of the deceased by communicating with the avatar of the deceased.

### CROSS-REFERENCE TO RELATED APPLICATION(S) AND CLAIM OF PRIORITY

This application is based on and claims priority under 35 U.S.C. § 119 to Korean Patent Application No. 10-2021-0181562, filed on Dec. 17, 2021, in the Korean Intellectual Property Office, the disclosure of which is herein incorporated by reference in its entirety.

### BACKGROUND

### Field

The disclosure relates to virtual reality (VR) and metaverse platform application technology, and more particularly, to a method and a system for operating a metaverse platform, which operate to remember activities of patients with physical difficulties and memories of the deceased.

### Description of Related Art

Some patients with physical difficulties may have a twisted facial expression or may stammer during a conversation, and may have difficulty in naturally expressing themselves, and accordingly, they may have a problem in communicating with their family or friends.

In addition, when a family or friends remember the deceased, they may remember the deceased with photos or videos, but there is a limitation thereto.

The metaverse refers to a three-dimensional virtual world through which user generated contents (UGC) created by users in the virtual world are distributed, and may not include only games or users' interactions performed in virtual reality (VR), and may also enable users to do social, cultural activities or to create economic values, and recently, interest therein is increasing.

Accordingly, there is a demand for a method for communicating with patients with physical difficulties or communicating with an avatar of the deceased by using metaverse technology.

### SUMMARY

To address the above-discussed deficiencies of the prior art, it is a primary object of the disclosure to provide a metaverse platform operating system and a method for communicating with patients with physical difficulties or communicating with an avatar of the deceased by using metaverse technology.

According to an embodiment of the disclosure to achieve the above-described objects, a metaverse platforming operating system may include: a data collection unit configured to collect image and voice data of a user; a voice and image correction module configured to correct an image and a voice of the user by inputting the collected image and voice data to a trained AI model; an avatar generation module configured to generate a virtual avatar reflecting a facial image of the user in a metaverse, based on the corrected image; and an avatar action control module configured to control an action of the generated avatar according to an inputted input signal.

In addition, the voice and image correction module may realize a figure of the user when the user was healthy, by correcting a figure corresponding to at least one of a decrepit/damaged figure of user's face, a twisted figure of the face, an injury to the face and body, a lean figure of the face and body, a skin tone, and hair loss, which are caused by a disease and a side effect of treatment of the user, in the collected image data.

In addition, the voice and image correction module may realize a conversation style that the user used when the user was healthy, by correcting a stammering portion of the user and restoring a sentence formed of a short sentence in the collected voice data.

In addition, the avatar action control module may trace user's pupils, may process a position change of the moving pupils as an input signal when the pupils move, and may control an action of the avatar.

In addition, when avatars of other users connect to the metaverse in addition to the avatar of the user and the user calls a name of a specific user, the avatar action control module may control the avatar of the user to approach an avatar of the called specific user, or may request the called specific user to approach the avatar of the user.

According to another embodiment of the disclosure, a metaverse platforming operating method may include: collecting, by a data collection unit, image and voice data of a user; correcting, by a voice and image correction module, an image and a voice of the user by inputting the collected image and voice data to a trained AI model; generating, by an avatar generation module, a virtual avatar reflecting a facial image of the user, based on the corrected image; and controlling, by an avatar action control module, an action of the generated avatar according to an inputted input signal.

In addition, according to another embodiment of the disclosure, a metaverse platform operating system may include: a data collection unit configured to collect lifetime image and voice data of the deceased; an avatar generation module configured to generate a virtual avatar reflecting a facial image of the deceased, based on the collected image data; and an AI responding module configured to control the avatar of the deceased, based on a trained AI model, to imitate behavior and a language habit that the deceased did during his/her lifetime in response to behavior and language of an avatar of the bereaved.

In addition, the AI responding module may include: a pattern extraction unit configured to extract a voice, a language habit, words, tones, intonation, behavior patterns of the deceased, based on the lifetime image and voice data of the deceased, which is collected through the data collection unit; and a pattern training unit configured to train the AI model for realizing behavior and conversation of the avatar by using the extracted patterns in order for the avatar to imitate the behavior and the language habit of the deceased.

In addition, the pattern training unit may input, to the AI model as training data, a plurality of script data for realizing a conversation according to a conversation style with a spouse, a child, a grandchild, a friend, or a specific acquaintance, and may convert a script of an appropriate flow into a voice imitating the voice of the deceased in response to a voice inputted from an avatar of a specific person, and may control the avatar of the deceased to output the voice.

In addition, the AI responding module may configure a metaverse environment based on data regarding a figure, a place, and object that the deceased liked during his/her lifetime.

In addition, according to an embodiment of the disclosure, the metaverse platform operating system may further include a record NFT module configured to make NFT data regarding a will, photos, images, voices, or other objects worth preserving, which are collected through the data collection unit, and to preserve the NFT data.

According to another embodiment of the disclosure, a metaverse platform operating method may include: collecting, by a data collection unit, lifetime image and voice data of the deceased; generating, by an avatar generation module, a virtual avatar reflecting a facial image of the deceased, based on the collected image data; and controlling, by an AI responding module, the avatar of the deceased, based on a trained AI model, to imitate behavior and a language habit that the deceased did during his/her lifetime in response to behavior and language of an avatar of the bereaved.

According to embodiments of the disclosure as described above, patients, family, and acquaintances may smoothly communicate with patients with physical difficulties in a metaverse virtual world, and may remember memories of the deceased by communicating with the avatar of the deceased.

Other aspects, advantages, and salient features of the invention will become apparent to those skilled in the art from the following detailed description, which, taken in conjunction with the annexed drawings, discloses exemplary embodiments of the invention.

Before undertaking the DETAILED DESCRIPTION OF THE INVENTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a view provided to explain a metaverse platform operating system according to a first embodiment of the disclosure;

FIG. 2 is a view provided to explain a metaverse platform operating method according to the first embodiment of the disclosure;

FIG. 3 is a view provided to explain a metaverse platform operating system according to a second embodiment of the disclosure;

FIG. 4 is a view provided to explain an artificial intelligence (AI) responding module in detail according to the second embodiment of the disclosure; and

FIG. 5 is a view provided to explain a metaverse platform operating method according to the second embodiment of the disclosure.

### DETAILED DESCRIPTION

Hereinafter, the disclosure will be described in more detail with reference to the accompanying drawings.

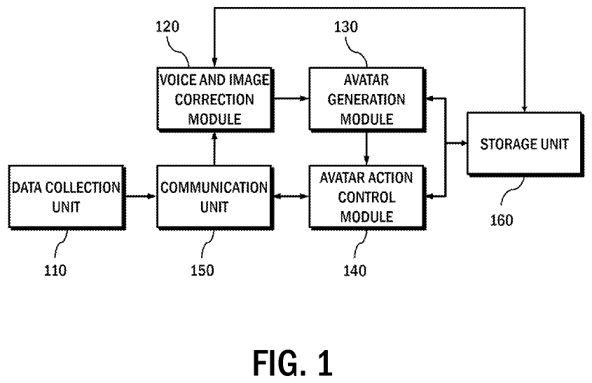

FIG. 1 is a view provide to explain a metaverse platform operating system according to a first embodiment of the disclosure.

The metaverse platform operating system according to the first embodiment may enable a family and acquaintances to smoothly communicate with patients with physical difficulties in a metaverse virtual world.

To achieve this, the metaverse platform operating system may include a data collection unit 110, a voice and image correction module 120, an avatar generation module 130, an avatar action control module 140, a communication unit 150, and a storage unit 160.

The data collection unit 110 may collect image and voice data of a user (patient).

Specifically, the data collection unit 110 may be implemented by a microphone and a camera which are mounted in a terminal, such as a head mounted display (HMD), VR glasses, a laptop, a smartphone, a tablet PC, or the like, and may collect image and voice data of a patient.

The voice and image correction module 120 may input the collected image and voice data to a trained AI model, and may correct an image and a voice of the user.

Specifically, the voice and image correction module 120 may realize a figure and a voice when the patient was healthy, by correcting stammering speech or a twisted face of the patient by using the AI learning model.

For example, the voice and image correction module 120 may realize a figure of the user when the user was healthy, by correcting a figure corresponding to at least one of a decrepit/damaged figure of user's face, a twisted figure of the face, an injury to the face and body, a lean figure of the face and body, a skin tone, and hair loss, which are caused by a disease and a side effect of treatment of the user in the collected image data, by using the AI learning model.

In addition, the voice and image correction module 120 may realize a voice (dialogic style) used when the user was healthy, by correcting user's stammering speech in the collected voice data and restoring a sentence formed of a short sentence.

The avatar generation module 130 may generate a virtual avatar reflecting a facial image when the user was healthy in the metaverse, based on the corrected image. In this case, the virtual avatar may connect to the metaverse world and communicate with avatars of patient's family and acquaintances while doing activities with them.

The avatar action control module 140 may control an action of the generated avatar according to an inputted input signal.

For example, the avatar action control module 140 may trace user's pupils in the facial image of the user which is collected through the data collection unit 110, and, when the pupils move, may process a position change of the moving pupils as an input signal and may control the action of the avatar.

In another example, when avatars of other users connect to the metaverse and the user calls the name of a specific user by a voice, the avatar action control module 140 may control the avatar of the user to approach the avatar of the called specific user or may request the called specific user to approach the avatar of the user.

Through this, an inconvenience that a patient with physical difficulties should operate a device with hand in order to control an action of the avatar may be solved.

The communication unit 150 may be connected with the data collection unit 110 provided in a terminal such as an HMD, VR glasses, a laptop, a smartphone, a tablet PC, or the like to collect image and voice data of the user.

In addition, the communication unit 150 may be wiredly or wirelessly connected to a terminal, a server, or a node which is necessary for a virtual avatar to connect to the metaverse and to do activities.

The storage unit 160 may be a storage medium that stores a program and data necessary for operating the voice and image correction module 120, the avatar generation module 130, and the avatar action control module 140.

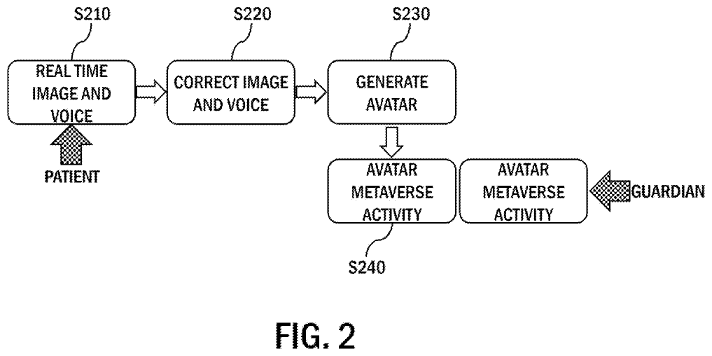

FIG. 2 is a view provided to explain a metaverse platform operating method according to the first embodiment of the disclosure.

The metaverse platform operating method according to the present embodiment may be executed by the metaverse platform operating system described above with reference to FIG. 1.

Referring to FIG. 2, the metaverse platform operating method may collect image and voice data of a user through the data collection unit 110 (S210), and, when the voice and image data are transmitted to the voice and image correction module 120, the voice and image correction module 120 may input the collected image and voice data to a trained AI model and may correct the user's image and voice (S220).

In addition, the metaverse platform operating method may generate a virtual avatar reflecting a facial image of the user through the avatar generation module 130, based on the corrected image (S230), and may control an action of the generated virtual avatar according to an inputted input signal through the avatar action control module 140.

Through this, a family and acquaintances may be enabled to smoothly communicate with the patient with physical difficulties in the metaverse virtual world.

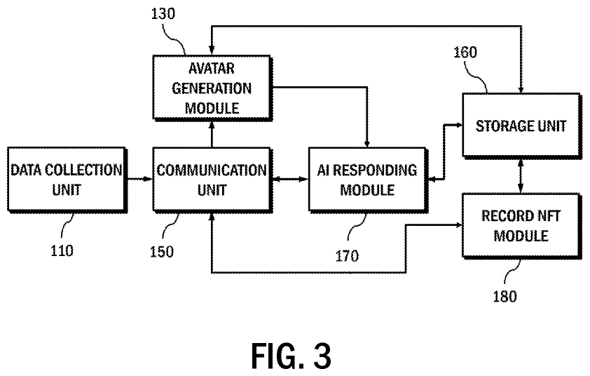

FIG. 3 is a view provided to explain a metaverse platform operating system according to a second embodiment of the disclosure.

The metaverse platform operating system according to the second embodiment may remember memories of the deceased by communicating with an avatar of the deceased.

To achieve this, the metaverse platform operating system may include a data collection unit 110, an avatar generation module 130, a communication unit 150, a storage unit 160, an AI responding module 170, and a record non-fungible token (NFT) module 180.

The data collection unit 110 may collect lifetime image and voice data of the deceased.

Specifically, the data collection unit 110 may be implemented by a microphone and a camera mounted in a terminal such as an HMD, VR glasses, a laptop, a smartphone, a tablet PC, or the like, and may collect lifetime image and voice data of the deceased.

The avatar generation module 130 may generate a virtual avatar reflecting a facial image of the deceased, based on the image data collected through the data collection unit 110.

The communication unit 150 may be connected with the data collection unit 110 provided in the terminal such as an HMD, VR glasses, a laptop, a smartphone, a tablet PC, or the like, and may collect user's image and voice data. In addition, the communication unit 150 may be wiredly or wirelessly connected to a terminal, a server, or a node which is necessary for the virtual avatar to connect to the metaverse and to do activities.

The storage unit 160 may be a storage medium which stores a program and data necessary for operations of the avatar generation module 130 and the AI responding module 170.

The AI responding module 170 may control the avatar of the deceased, based on a trained AI model, to imitate behavior and a language habit of the deceased in response to behavior and language of an avatar of the bereaved.

In addition, the AI responding module 170 may configure a metaverse environment, based on data regarding a figure, a place, and an object that the deceased liked during his/her lifetime.

The record NFT module 180 may make NFT data regarding a will, photos, images, voices, or other objects worth preserving, which are collected through the data collection unit 110, and may preserve the data.

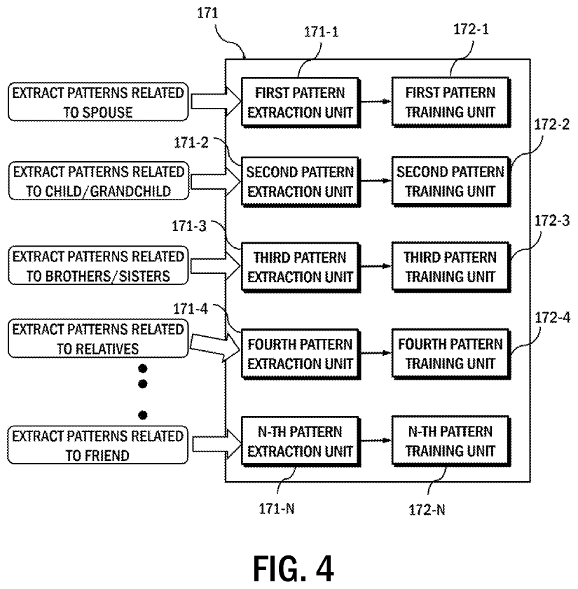

FIG. 4 is a view provided to explain the AI responding module 170 in detail according to the second embodiment.

Referring to FIG. 4, the AI responding module 170 may include a pattern extraction unit 171 to extract a voice, a language habit, words, tones, intonation, behavior patterns of the deceased, based on the lifetime image and voice data of the deceased, which is collected through the data collection unit 110, and a pattern training unit 172 to train the AI model for realizing behavior and conversation of an avatar by using the extracted patterns, and to control the avatar to imitate behavior and a language habit of the deceased.

In addition, a plurality of pattern extraction units 171 may be provided, and the plurality of pattern extraction units 171-1 to 171-N may individually extract respective patterns according to a conversation style and a behavior style of the deceased with other people. Herein, the other people may be a spouse, a child, a grandchild, a friend, or an acquaintance.

For example, the first pattern extraction unit 171-1 may extract a voice, a language habit, words, tones, intonation, behavior patterns of the deceased when he/she faces the spouse, based on voice and image data related to conversation between the deceased and the spouse.

In addition, the second pattern extraction unit 171-2 may extract a voice, a language habit, words, tones, intonation, behavior patterns of the deceased when he/she faces a child or grandchild, based on voice and image data related to conversation between the deceased and the child or grandchild of the deceased.

Regarding brothers/sisters and a specific acquaintance, unique patterns may be extracted through the respective pattern extraction units 171-1 to 171-N in the above-described way.

That is, the pattern extraction units 171-1 to 171-N may input, to the AI model as training data, a plurality of script data including patterns extracted according to a conversation style with the spouse, child, grandchild, friend or a specific acquaintance, and may train the AI model.

In this case, the trained AI model may convert a script of an appropriate flow into a voice imitating the voice of the deceased in response to a voice inputted by an avatar of a specific person, and may control the avatar of the deceased to output the voice.

In addition, a plurality of pattern training units 172 may be provided, and the plurality of pattern training units 172 may be matched with the pattern extraction units 171-1 to 171-N one to one, and may train the AI model for realizing behavior and conversation of the avatar by using unique patterns related to a specific person, which are extracted by the pattern extraction units 171-1 to 171-N.

That is, when the avatar of the deceased faces a virtual avatar of the spouse, behavior and conversation of the avatar may be realized through a first AI model which learns patterns related to the spouse, and, when the avatar of the deceased faces a virtual avatar of a child or grandchild of the deceased, behavior and conversation of the avatar may be realized through a second AI model which learns patterns related to the child or grandchild of the deceased.

Through this, a virtual avatar is not realized simply based on voices and images of the deceased, and unique patterns may be applied according to whom the avatar faces, so that virtual avatars of the bereaved may communicate with the avatar of the deceased which is realized more realistically and in more detail.

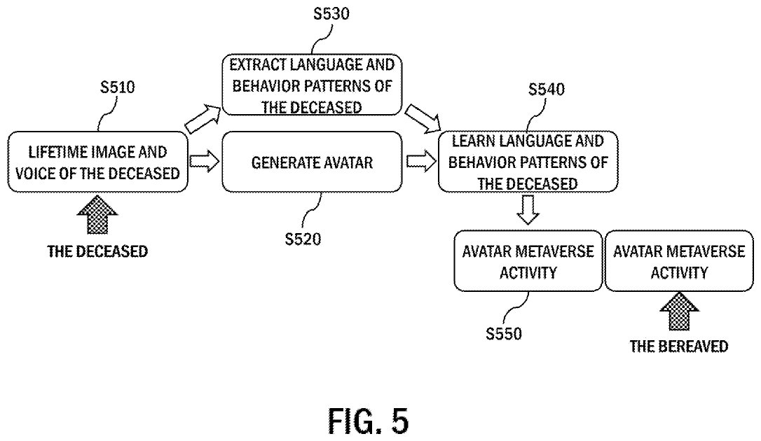

FIG. 5 is a view provided to explain a metaverse platform operating method according to the second embodiment of the disclosure.

The metaverse platform operating method according to the present embodiment may be executed by the metaverse platform operating system described above with reference to FIGS. 3 and 4.

Referring to FIG. 5, the metaverse platform operating method may collect lifetime image and voice data of the deceased through the data collection unit 110 (S510), and may transmit the image and voice data to the avatar generation module 130, and the avatar generation module 130 may generate a virtual avatar reflecting a facial image of the deceased, based on the collected image data (S520).

The metaverse platform operating method may extract a voice, a language habit, words, tones, intonation, behavior patterns of the deceased, based on the lifetime image and voice data of the deceased, which is collected through the data collection unit 110 (S530), and may train the AI model for realizing behavior and conversation of the avatar based on the extracted patterns (S540).

When training of the AI model for realizing the behavior and conversation of the avatar is completed, the metaverse platform operating method may control the avatar of the deceased based on the trained AI model, to imitate behavior and conversation that the deceased did for his/her lifetime in response to behavior and language of an avatar of the bereaved (S550).

Through this, memories of the deceased may be remembered through communication with the avatar of the deceased.

The technical concept of the present disclosure may be applied to a computer-readable recording medium which records a computer program for performing the functions of the apparatus and the method according to the present embodiments. In addition, the technical idea according to various embodiments of the present disclosure may be implemented in the form of a computer readable code recorded on the computer-readable recording medium. The computer-readable recording medium may be any data storage device that can be read by a computer and can store data. For example, the computer-readable recording medium may be a read only memory (ROM), a random access memory (RAM), a CD-ROM, a magnetic tape, a floppy disk, an optical disk, a hard disk drive, or the like. A computer readable code or program that is stored in the computer readable recording medium may be transmitted via a network connected between computers.

In addition, while preferred embodiments of the present disclosure have been illustrated and described, the present disclosure is not limited to the above-described specific embodiments. Various changes can be made by a person skilled in the art without departing from the scope of the present disclosure claimed in claims, and also, changed embodiments should not be understood as being separate from the technical idea or prospect of the present disclosure.

What is claimed is:

1. A metaverse platforming operating system comprising:
a data collection unit configured to collect image and voice data of a user;
a voice and image correction module configured to correct an image and a voice of the user by inputting the collected image and voice data to a trained AI model;
an avatar generation module configured to generate a virtual avatar reflecting a facial image of the user in a metaverse, based on the corrected image; and
an avatar action control module configured to control an action of the generated avatar according to an inputted input signal.

2. The metaverse platform operating system of claim 1, wherein the voice and image correction module is configured to realize a figure of the user when the user was healthy, by correcting a figure corresponding to at least one of a decrepit/damaged figure of user's face, a twisted figure of the face, an injury to the face and body, a lean figure of the face and body, a skin tone, and hair loss, which are caused by a disease and a side effect of treatment of the user, in the collected image data.

3. The metaverse platform operating system of claim 1, wherein the voice and image correction module is configured to realize a conversation style that the user used when the user was healthy, by correcting a stammering portion of the user and restoring a sentence formed of a short sentence in the collected voice data.

4. The metaverse platform operating system of claim 1, wherein the avatar action control module is configured to trace user's pupils, to process a position change of the moving pupils as an input signal when the pupils move, and to control an action of the avatar.

5. The metaverse platform operating system of claim 1, wherein the avatar action control module is configured to, when avatars of other users connect to the metaverse in addition to the avatar of the user and the user calls a name of a specific user, control the avatar of the user to approach an avatar of the called specific user, or request the called specific user to approach the avatar of the user.

6. A metaverse platforming operating method comprising:
collecting, by a data collection unit, image and voice data of a user;
correcting, by a voice and image correction module, an image and a voice of the user by inputting the collected image and voice data to a trained AI model;
generating, by an avatar generation module, a virtual avatar reflecting a facial image of the user, based on the corrected image; and
controlling, by an avatar action control module, an action of the generated avatar according to an inputted input signal.

7. A metaverse platform operating system comprising:
a data collection unit configured to collect lifetime image and voice data of the deceased;
an avatar generation module configured to generate a virtual avatar reflecting a facial image of the deceased, based on the collected image data; and
an AI responding module configured to control the avatar of the deceased, based on a trained AI model, to imitate behavior and a language habit that the deceased did during his/her lifetime in response to behavior and language of an avatar of the bereaved.

8. The metaverse platform operating system of claim 7, wherein the AI responding module comprises:
a pattern extraction unit configured to extract a voice, a language habit, words, tones, intonation, behavior patterns of the deceased, based on the lifetime image and voice data of the deceased, which is collected through the data collection unit; and
a pattern training unit configured to train the AI model for realizing behavior and conversation of the avatar by using the extracted patterns in order for the avatar to imitate the behavior and the language habit of the deceased.

9. The metaverse platform operating system of claim 8, wherein the pattern training unit is configured to input, to the AI model as training data, a plurality of script data for realizing a conversation according to a conversation style with a spouse, a child, a grandchild, a friend, or a specific acquaintance, and to convert a script of an appropriate flow into a voice imitating the voice of the deceased in response to a voice inputted from an avatar of a specific person, and to control the avatar of the deceased to output the voice.

10. The metaverse platform operating system of claim 7, wherein the AI responding module is configured to configure a metaverse environment based on data regarding a figure, a place, and object that the deceased liked during his/her lifetime.

11. The metaverse platform operating system of claim 7, further comprising a record NFT module configured to make NFT data regarding a will, photos, images, voices, or other objects worth preserving, which are collected through the data collection unit, and to preserve the NFT data.
